(19) United States
(12) Patent Application Publication
(10) Pub. No.: US 2022/0249879 A1
(43) Pub. Date: Aug. 11, 2022

(54) ORO-NASAL INTERFACE DEVICE INCLUDING A FILTRATION AND DISINFECTION SYSTEM

(71) Applicant: Chicago Controls Thermostats, Inc., Chicago, IL (US)

(72) Inventor: Michael Lyle, Chicago, IL (US)

(21) Appl. No.: 17/667,198

(22) Filed: Feb. 8, 2022

Related U.S. Application Data
(60) Provisional application No. 63/147,014, filed on Feb. 8, 2021.

(51) Int. Cl. A62B 7/10; A62B 18/00; A62B 7/12; A61L 9/20; A41D 13/11

(52) U.S. Cl. CPC A62B 7/10; A62B 18/003; A61L 2209/14; A61L 9/20; A41D 13/11; A62B 7/12

(57) ABSTRACT

An oro-nasal interface device includes: a facemask; an intake tube with an inner surface made from a reflective material; an exhaust tube; an intake disinfection unit located at a first end of the intake tube, the intake disinfection unit housing: a battery-powered intake fan in fluid communication with the intake tube that, in operation, creates a positive pressure air flow through the intake tube into the facemask; an intake high-efficiency particulate air filter in fluid communication with the intake tube that filters air passing through the intake tube prior to reaching the facemask; and an intake UV radiation source acting on the air passing through the intake tube prior to reaching the facemask; and an exhaust high-efficiency particulate air filter in fluid communication with the exhaust tube that filters air passing through the exhaust tube after leaving the facemask.

### CROSS REFERENCE TO RELATED APPLICATIONS

The present application claims the benefit of priority to U.S. Application No. 63/147,014 filed Feb. 8, 2021, the entire contents of which are being incorporated herein by reference.

### BACKGROUND

The invention relates to an oro-nasal interface device, such as a facemask, incorporating a filtration and disinfection system. The oro-nasal interface device can protect the user from infection with airborne pathogens.

In the event of respiratory virus pandemics, such as influenza virus or coronavirus, and in the absence of and/or in addition to effective vaccines and antiviral drugs, blocking the spread of virus has been one of the most effective strategies recommended to protect the public. In general, the spread of respiratory viral infections occurs mainly through contact and droplet routes. However, there is evidence that suggests some viruses, such as severe acute respiratory coronavirus can remain infectious in aerosol for hours.

Facemasks are recommended for diseases transmitted through air droplets. The filtration provided by the facemasks, whether simply cloth masks, those incorporating specific filters such as HEPA filters, or N95 filtering facepiece respirators (N95s), act as a barrier to transmission of air droplets. Yet there are unmet needs for facemasks to provide more efficient protection against infectious diseases, especially those that can be transmitted through respiratory aerosols.

Ultraviolet (UV) radiation has been shown to effectively kill viruses and bacteria in the air, in liquids, or on surfaces. UV radiation is commonly used inside air ducts to disinfect the air. It is considered the safest way to employ UV radiation to avoid direct UV exposure to human skin or eyes that may cause injuries.

Accordingly, there is a need for a facemask, with or without a filtration system, including a disinfection unit employing UV radiation to provide sufficient protection against infection with airborne pathogens, as provided herein.

### SUMMARY

The present disclosure provides a facemask including a filtration and disinfection system that is used to filter and disinfect the air to help protect the user from inhaling pathogens as well as to filter and disinfect the air exhaled by the user before returning it to the surrounding environment. In a primary embodiment, the filtration and disinfection system further includes a positive pressure air source providing air to the user through the facemask. A first disinfection unit may act on the air provided to the facemask by the positive pressure air source and a second disinfection unit may act on the air exhaled by the user that passes through the facemask back into the surrounding environment. The facemask may cover the user's nose and mouth, may additionally cover the user's eyes, or may even cover a greater portion of the user's face and head. The facemask may be clear so people with whom the user is engaged can see through the mask, which may help improve communications and the ability for others to read facial expressions when the mask is worn.

Each of the first and second disinfection units may include both a filter element and a disinfection element. For example, in the intake disinfection unit, the filter element may be a high-efficiency particulate air (HEPA) filter positioned on/in the disinfection unit itself and/or an intake tube through which air enters the facemask. Similarly, the exhaust disinfection unit may include a HEPA filter positioned on/in the disinfection unit itself and/or an exhaust tube through which air leaves the facemask. UV light may be used to disinfect the air within the intake and/or exhaust tubes. The intake and exhaust tubes may be coated on the interior to be internally reflective to increase the effectiveness of the UV light reflecting within.

In some embodiments, the facemask covers the oral and nasal area of the user and receives positive pressure airflow from an intake disinfection unit that is a small, portable, battery-powered unit that includes a filter, a UV light source, and an intake fan. In operation, air is drawn through the filter by the fan and supplied to the facemask through the intake tube. The disinfection unit includes a UV light source that acts on the air flowing from the intake disinfection unit to the facemask. Accordingly, in operation, the air that is provided to the facemask for the user to breathe is first filtered and then disinfected before being exposed to the user.

In some embodiments, the facemask exhausts into an exhaust disinfection unit. Like the intake disinfection unit described above, the exhaust disinfection unit may include a filter and a UV light source. In some embodiments, it may also include an exhaust fan. In use, the air exhaled by the user passes into the exhaust tube, is acted on by the UV light source to disinfect the air, and then passes through the filter before being returned to the ambient environment. In some embodiments, the system may include both an intake disinfection unit with an intake fan and an exhaust disinfection unit with an exhaust fan. In some embodiments, there is only an intake fan. In some embodiments, there is only an exhaust fan. In some embodiments, there are no fans.

In an alternative embodiment, rather than pulling air from the ambient environment, the intake disinfection unit may be connected to a positive pressure air supply, such as, for example, the air supply associated with a seat on a commercial flight or provided on public transportation such as a bus or a train. In such embodiments, the intake fan may be unused, disabled, or simply not included in the unit.

In one example, an oro-nasal interface device includes a facemask covering the nose and mouth of a user, an intake tube in fluid communication with the facemask, an exhaust tube in fluid communication with the facemask, an air source in fluid communication with the intake tube configured to create a positive pressure air flow through the intake tube into the facemask; an intake air filter in fluid communication with the intake tube configured to filter the positive pressure air flow passing through the intake tube prior to reaching the facemask; and an intake UV radiation source acting on the positive pressure air flow passing through the intake tube prior to reaching the facemask.

The air filters may be high-efficiency particulate air filters. The air source, the intake air filter, and the intake UV radiation source may be housed within an intake disinfection unit located at a first end of the intake tube opposite to a second end of the intake tube which is attached to the facemask. The air source may be a battery-powered intake fan. The inner surfaces of the intake tube may be made from a reflective material. The device may further include an air filter in fluid communication with the exhaust tube that filters air passing through the exhaust tube after exhausting from the facemask. The device may further include an exhaust UV radiation source acting on the air passing through the exhaust tube after leaving the facemask and the inner surface of the exhaust tube may be made from a reflective material.

In another example, an oro-nasal interface device includes: a facemask covering the nose and mouth of a user; an intake tube in fluid communication with the facemask; an exhaust tube in fluid communication with the facemask; an air source in fluid communication with the intake tube configured to create a positive pressure air flow through the intake tube into the facemask; an intake air filter in fluid communication with the intake tube that filters air passing through the intake tube prior to reaching the facemask; and an exhaust air filter in fluid communication with the exhaust tube that filters air passing through the exhaust tube after leaving the facemask.

The device may further include an intake UV radiation source acting on the air passing through the intake tube prior to reaching the facemask. The intake air filter and the exhaust air filter may each be high-efficiency particulate air filters. The air source and the intake air filter may be housed within an intake disinfection unit located at a first end of the intake tube opposite to a second end of the intake tube which is attached to the facemask. The air source may be a battery-powered intake fan. The intake disinfection unit further may further include an intake UV radiation source acting on the air passing through the intake tube prior to reaching the facemask. The inner surface of the intake tube may be made from a reflective material. The device may further include an exhaust UV radiation source acting on the air passing through the exhaust tube after leaving the facemask and the inner surface of the exhaust tube may be made from a reflective material.

In yet another example, an oro-nasal interface device includes: a facemask covering the nose and mouth of a user; an intake tube in fluid communication with the facemask, wherein the inner surface of the intake tube comprises a reflective material; an exhaust tube in fluid communication with the facemask; an intake disinfection unit located at a first end of the intake tube opposite to a second end of the intake tube which is attached to the facemask, the intake disinfection unit comprising: a battery-powered intake fan in fluid communication with the intake tube that, in operation, creates a positive pressure air flow through the intake tube into the facemask; an intake high-efficiency particulate air filter in fluid communication with the intake tube that filters air passing through the intake tube prior to reaching the facemask; and an intake UV radiation source acting on the air passing through the intake tube prior to reaching the facemask; and an exhaust high-efficiency particulate air filter in fluid communication with the exhaust tube that filters air passing through the exhaust tube after leaving the facemask.

The device may further include an exhaust UV radiation source acting on the air passing through the exhaust tube after leaving the facemask, wherein the inner surface of the exhaust tube comprises a reflective material.

One object of the invention is to provide protection against airborne viruses, bacteria, and other infectious disease in public settings, including public transportation in which a group of people are getting air from a common air source.

One advantage to the invention is that the filtration and disinfection system eliminates airborne pathogens in the air before the user inhales them.

A further benefit of the invention is that it may both passively filter the air and actively destroy pathogens using UV radiation.

Another advantage of the invention is that the device is portable and the intake and/or exhaust disinfection units may be compact enough for the user to carry or wear the units.

Another advantage of the invention is that the device can be attached to the air supply source. This allows the application of the device to the public transportation setting, such as on an airplane.

Other aspects, objects and advantages of the invention will become apparent from reading the description of a number of these embodiments.

### BRIEF DESCRIPTION OF THE DRAWINGS

FIG. 1 is an example of an oro-nasal interface device of the present application.

FIG. 2A is a cross-sectional view of the disinfection unit taken generally along lines A-A of the oro-nasal interface device shown in FIG. 1.

FIG. 2B is a cross-sectional view of the intake tube taken generally along lines B-B of the oro-nasal interface device shown in FIG. 1.

FIG. 3 is an exploded view of the intake and exhaust tubes on the facemask of the oro-nasal interface device shown in FIG. 1.

FIG. 4 is a further embodiment the oro-nasal interface device including an eye covering.

FIG. 5 is a still further embodiment the oro-nasal interface device including a full facemask.

FIG. 6 is a further embodiment the oro-nasal interface device including a second disinfection unit on the exhaust tube.

### DETAILED DESCRIPTION

The present disclosure provides for a protective oro-nasal interface device 100 including a facemask 102 that covers both the nose and the mouth of a user and a disinfection unit 104 that is used to disinfect the air before the user inhales the air through facemask 102. Such devices 100 will be useful for protecting the user from airborne pathogens.

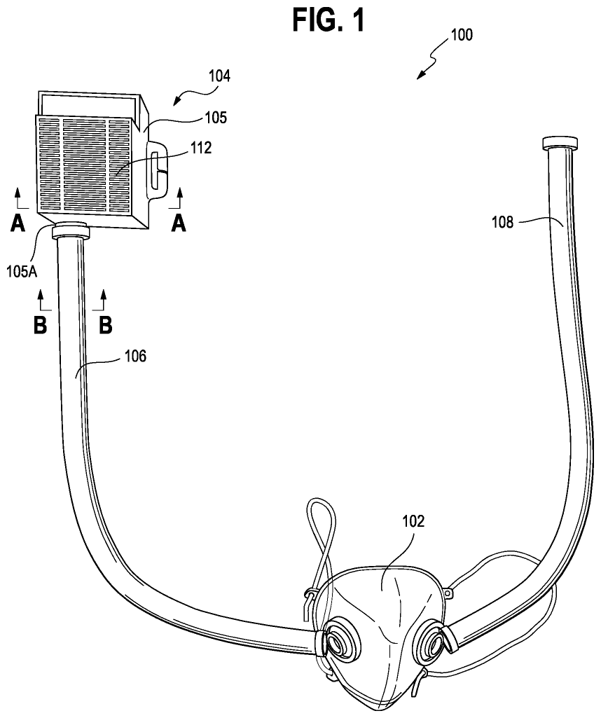

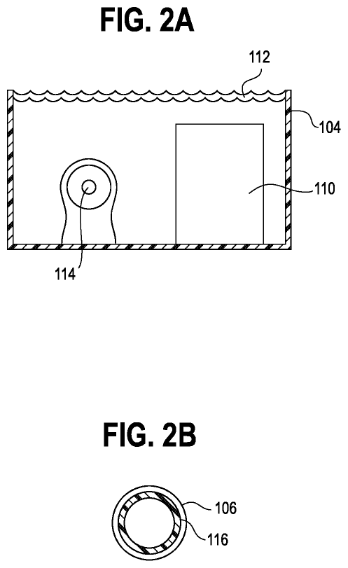

As shown in FIGS. 1-6, the device 100 includes a facemask 102 covering the nose and mouth of a user, an intake tube 106 in fluid communication with the facemask 102 and an exhaust tube 108 in fluid communication with the facemask 102. Referring to FIG. 2A, the disinfection unit 104 includes a housing 105 within which an air source (e.g., an intake fan) 110 is positioned. The intake tube 106 is secured to an outlet port 105A of the housing 105. The air source 110 is in fluid communication with the intake tube 106 such that, in operation, the air source 110 creates a positive pressure air flow through the intake tube 106 into the facemask 102.

An intake air filter 112 is provided along a portion of the housing 105 of the disinfection unit 104, as seen in FIGS. 1 and 2A. The air filter 112 filters air passing entering the disinfection unit 104 and passing through to the intake tube 106. In other embodiments, the disinfection unit 104 may comprise a solid housing structure with a hole or opening through which air is pulled into the air source 110, and the air filter 112 may be positioned across the opening upstream of the air source 110 and/or downstream of the air source 110 before air is directed out of the outlet port 105A and into the intake tube 106.

An intake UV radiation source 114 also acts on the air passing through the intake tube 106 prior to reaching the facemask 102. In the illustrated embodiment, the UV radiation source 114 is a UV light source positioned within the disinfection unit 104 so that it directs UV light into the center of the intake tube 106 at the outlet port 105A. In other embodiments, the UV radiation source 114 may be a ring of UV light sources mounted within the intake tube 106 directed in one or multiple directions. Preferably, the UV radiation source 114 includes one or more UVC lamps. The UV radiation source 114 may emit ultraviolet radiation, such as ultraviolet-C radiation, in sufficient amounts to disinfect the air moving through the disinfection unit 104. The UV radiation source 114 may be battery-powered and operated through a switch on the housing of the disinfection unit. In still further embodiments, the disinfection unit 104 may include a UV radiation source 114 without an air filter 112 for disinfecting air passing through the intake tube 106 before reaching the facemask 102.

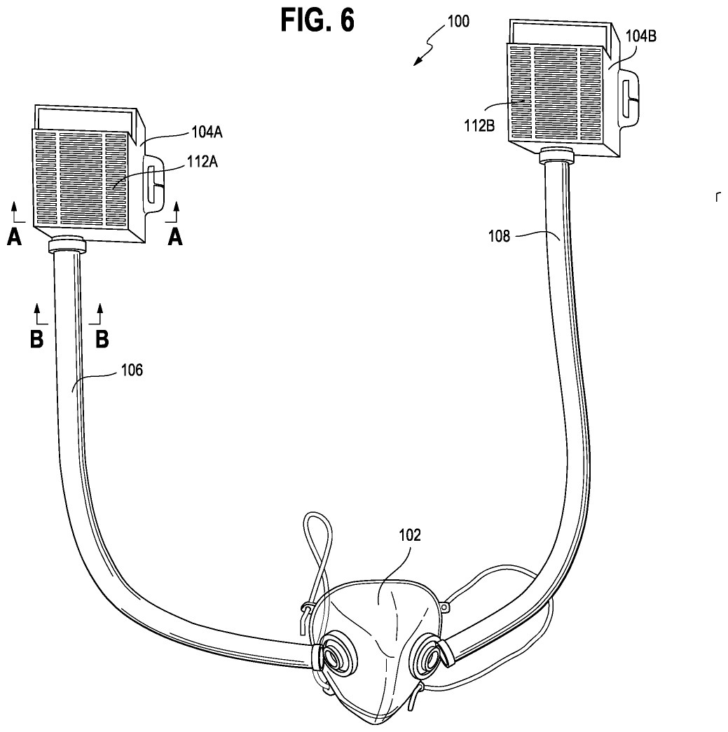

In FIG. 1, the intake fan 110, the intake air filter 112, and the UV radiation source 114 are housed in the battery-powered intake disinfection unit 104. In another example embodiment shown in FIG. 6, the device 100 may also include a first disinfection unit 104A on the intake tube 106 and a second disinfection unit 104B on the exhaust tube 108. The second disinfection unit 104B may include a second filter 112B and/or a second exhaust UV radiation source positioned similarly to the UV radiation source 114 as shown in FIG. 2A acting on the exhaust tube 108 instead of the intake tube 106 to disinfect the exhaled air before it returns to the ambient environment.

The disinfection unit 104 utilizes UV radiation to disinfect airborne pathogen, such as influenza virus and coronavirus. In the device 100 shown in the FIGS. 1-6, the combination of the air filter 112 and the UV radiation source 114 disinfect the air as it passes through the disinfection unit 104 and travels through the intake tube 106. The presently disclosed device 100 may be more protective than a regular cloth facemask because it not only blocks the airborne pathogen, but also kills the pathogen by UV radiation.

In some embodiments, the intake and exhaust tubes 106, 108 comprise a translucent material. Preferably, the inner surface of the intake tube 106 comprises a reflective material 116 such that any UV radiation in the tube 106 reflects back into and along the length of the tube 106, thereby increasing its effectiveness in killing pathogens in the tube 106. For example, as illustrated in FIG. 2B, the tube 106 may comprise an inner coating of reflective material 116, such as a reflective tape. Examples of reflective material include but are not limited to coatings and/or tape that include aluminum, stainless steel, and polytetrafluoroethylene (PTFE).

The facemask 102 may comprises a clear plastic material so that users can view the faces of other users, allowing users to express and understand facial expressions that is not available through the use of a N95 or cloth face mask.

In some examples, the presently disclosed device 100 connects to an external air supply source. For example, an external air supply, such as an air supply associated with a commercial airline seat or within a bus or other public transportation, may connect directly to the intake disinfection unit 106. In such embodiments, the intake air source 110 may be omitted or inactive.

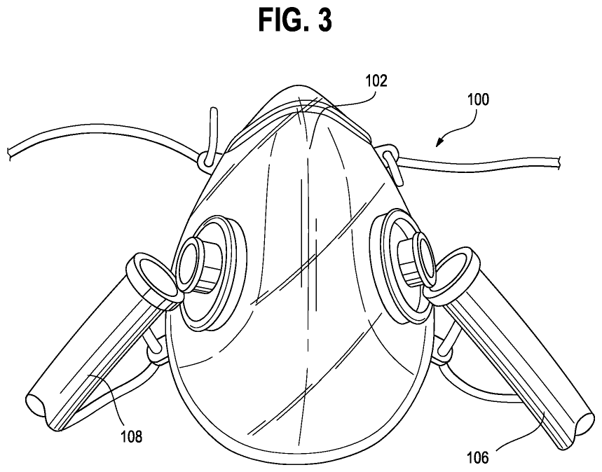

FIG. 3 illustrates that the elements of the device 100 such as the facemask 102, the intake tube 106, and the exhaust tube 108, may be removably connected such that they may each be individually cleaned or replaced. The fittings connecting the tubes 106, 108 to the facemask 102 shown in FIG. 3 are friction fittings, which is only one example of removable fittings that may be used in the device.

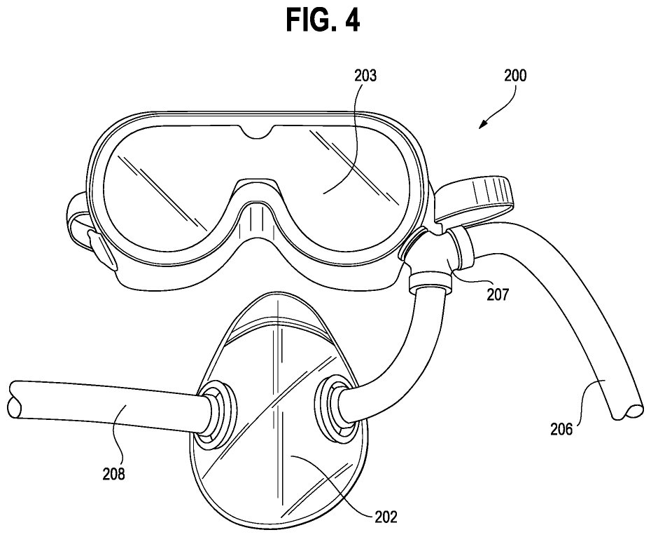

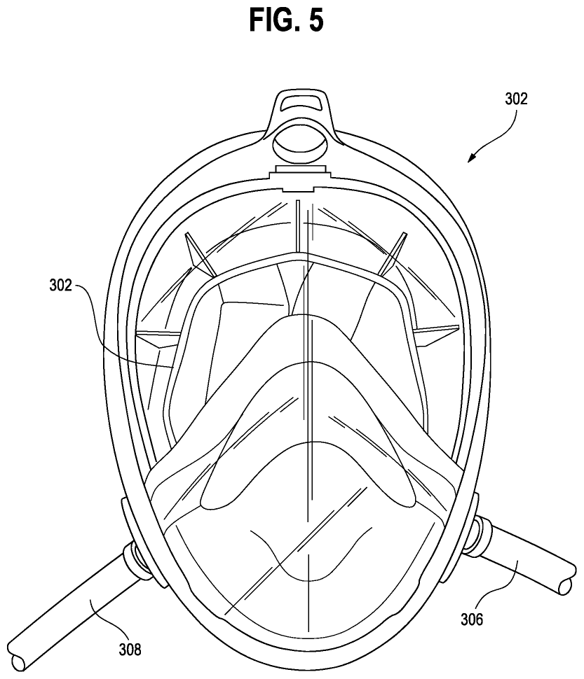

FIG. 4 illustrates another example of an oro-nasal interface device 200 including an eye covering 203, such as googles. The intake tube 206 includes a t-shaped connector 207 that allows the tube 206 to connect to the eye covering 203 through a removable fitting before the tube 206 connects to the nasal facemask 202. In the further embodiment illustrated in FIG. 5, the facemask 302 of the oro-nasal interface device 300 is a full face mask integrating the eye protection with the nose and mouth protection.

Without further elaboration, it is believed that one skilled in the art can use the preceding description to utilize the claimed inventions to their fullest extent. The examples and embodiments disclosed herein are to be construed as merely illustrative and not a limitation of the scope of the present disclosure in any way. It will be apparent to those having skill in the art that changes may be made to the details of the above-described embodiments without departing from the underlying principles discussed. In other words, various modifications and improvements of the embodiments specifically disclosed in the description above are within the scope of the appended claims. For example, any suitable combination of features of the various embodiments described is contemplated.

The invention is claimed as follows:

1. An oro-nasal interface device comprising:
a facemask covering the nose and mouth of a user;
an intake tube in fluid communication with the facemask;
an air source configured to create a positive pressure air flow through the intake tube into the facemask;
an air filter configured to filter the positive pressure air flow passing through the intake tube; and
a UV radiation source configured to disinfect the positive pressure air flow passing through the intake tube.

2. The device of claim 1, wherein the air filter is a high-efficiency particulate air filter.

3. The device of claim 1, wherein the disinfection unit is located at a first end of the intake tube opposite to a second end of the intake tube, the second end of the intake tube being attached to the facemask.

4. The device of claim 3, wherein the air source is a battery-powered intake fan.

5. The device of claim 3, wherein the air filter is a high-efficiency particulate air filter.

6. The device of claim 1, wherein an inner surface of the intake tube includes a reflective material.

7. The device of claim 1, further comprising an exhaust tube and an additional air filter in fluid communication with the exhaust tube, the additional air filter configured to filter air passing through the exhaust tube.

8. The device of claim 7, further comprising a further UV radiation source configured to disinfect the air passing through the exhaust tube.

9. The device of claim 8, wherein an inner surface of the exhaust tube includes a reflective material.

10. An oro-nasal interface device comprising:
a facemask covering the nose and mouth of a user;
an intake tube in fluid communication with the facemask; and
a disinfection unit in fluid communication with the intake tube, the disinfection unit comprising:
an air source configured to create a positive pressure air flow through the intake tube into the facemask;
an air filter configured to filter the positive pressure air flow passing through the intake tube; and
a UV radiation source acting on the air passing through the intake tube prior to reaching the facemask.

11. The device of claim 10, wherein the air filter is a high-efficiency particulate air filter.

12. The device of claim 10, wherein an inner surface of the intake tube comprises a reflective material.

13. The device of claim 10, further comprising an exhaust tube and a further disinfection unit, the further disinfection unit comprising:
a further air filter configured to filter air passing through the exhaust tube; and
a further UV radiation source configured to disinfect the air passing through the exhaust tube.

14. The device of claim 13, wherein an inner surface of the exhaust tube includes a reflective material.

15. An oro-nasal interface device comprising:
a facemask covering the nose and mouth of a user;
an intake tube in fluid communication with the facemask, wherein the inner surface of the intake tube comprises a reflective material;
an exhaust tube in fluid communication with the facemask;
a battery-powered intake fan in fluid communication with the intake tube configured to create a positive pressure air flow through the intake tube into the facemask;
an intake high-efficiency particulate air filter in fluid communication with the intake tube configured to filter the positive pressure air flow passing through the intake tube prior to reaching the facemask;
an intake UV radiation source acting on the positive pressure air flow passing through the intake tube prior to reaching the facemask; and
an exhaust high-efficiency particulate air filter in fluid communication with the exhaust tube configured to filter air passing through the exhaust tube after leaving the facemask.

16. The device of claim 16, further comprising an exhaust UV radiation source configured to disinfect the air passing through the exhaust tube after leaving the facemask, wherein the inner surface of the exhaust tube comprises a reflective material.
